(19) United States
(12) Patent Application Publication
(10) Pub. No.: US 2005/0251081 A1
(43) Pub. Date: Nov. 10, 2005

(54) Orthotic foot care and platform method and apparatus

(76) Inventors: Raymond A. McClanahan, Scappoose, OR (US); Mark E. Kelch, Hood River, OR (US)

Correspondence Address: IDEA ADVOCATES LAW GROUP LLC, 239 NW 13TH AVE., SUITE 310, PORTLAND, OR, 97209, US

(21) Appl. No.: 10/828,661

(22) Filed: Apr. 21, 2004

(51) Int. Cl. A61F 5/00; A63B 23/16

(52) U.S. Cl. 602/30

(57) ABSTRACT

The invention involves foot care method and apparatus for imposing a predetermined inter-digital spacing between adjacent ones of one or more pairs of toes on a foot for purposes of correcting deformed joints and/or appendages. The apparatus includes one or more inter-digital columns extending between the toes, each column being width-adjustable to fix inter-digital spacing. The foot care method involves fitting a user's foot with such apparatus and adjusting the width of the one or more inserts to properly splay the user's toes as indicated by the location and extent of the deformation. The apparatus is gripped by the toes of the user's foot or, alternatively, may be suitably fastened.

### BACKGROUND OF THE INVENTION

The invention relates generally to orthotic foot care, by which is meant therapies and appliances for straightening crooked joints or appendages. More particularly, it concerns the provision of orthotic foot care and apparatus for splaying the toes of a human's foot to restore the foot's joints to a more natural and healthy configuration.

Human feet are deformed by wearing modern shoes and boots and even socks, which confine the toes within a vertical and horizontal space, i.e. the so-called ‘toe box’, that unnaturally forces the toes into lateral proximity, contact and impingement. Foot problems abound, as a result of wearing traditional shoes or boots or even socks. Such problems include bunions, hammertoes, ingrown toenails and other problems too numerous to recount. In shoeless cultures, foot problems affect only approximately 3% of the population, whereas in shoe-wearing cultures, foot problems affect nearly 75% of the population.

Many shoes or boots are simply ill-fitted, i.e. they are fitted either too small or too narrow for the wearer's foot. Some of the reasons for such ill-fitting shoes include buying pressure, bad sales technique, lack of time, skill or knowledge, or bad design or manufacture. Self-image impacts many shoe buying decisions, and it is modern western prejudice to purchase narrow-toed, small shoes to meet societal pressure to conform to fashion and size trends. Modern shoes and boots conform the human foot to a deformed configuration that is a far cry from the natural form of a newborn baby's unshod foot.

### SUMMARY

The invention involves foot care method and apparatus for imposing an adjustably predetermined (adjustable once and then relatively fixed) inter-digital spacing between adjacent ones of one or more pairs of toes for purposes of correcting deformed joints and/or appendages. The apparatus includes one or more inter-digital columns extending between the toes, each column having a recess therein receiving a variable width insert, with preferably plural columns being fixedly attached to one or more upper and lower interconnecting web structures that position the plural columns to fit between the toes. The foot care method involves fitting a user's foot with such apparatus and adjusting the width of the one or more inserts to properly splay the user's toes as indicated by the location and extent of the deformation. Preferably, the columns are integrally molded with the web structures of an elastomeric material and the inserts are formed of a rigid foam material that rigidizes the columns and fixes the toe spacing. Alternatively, the columns may take the form of a pneumatic or hydraulic, pressurizable bladder that, alone or in combination with a surrounding spacer, adjusts the inter-digital spacing to a desired spacing. The apparatus is gripped by the toes of the user's foot or, alternatively, may be suitably fastened.

### BRIEF DESCRIPTION OF THE DRAWINGS

FIG. 1 is an isometric view of the orthotic apparatus in accordance with one embodiment of the invention.

FIG. 2 is an isometric view of the orthotic apparatus in accordance with another embodiment of the invention.

FIGS. 3A and 3B are enlarged detailed fragmentary cutaway isometric views of one of the columns of the apparatus shown in FIG. 1, FIG. 3A showing an elastomeric shim inserted within a recess and FIG. 3B showing a pneumatic bladder inserted therein.

FIGS. 4A and 4B are schematic diagrams illustrating a foot respectively without and with the use of the invented orthotic method and apparatus.

FIG. 5 is a flowchart illustrating an orthotic foot care method in accordance with the invention.

### DETAILED DESCRIPTION

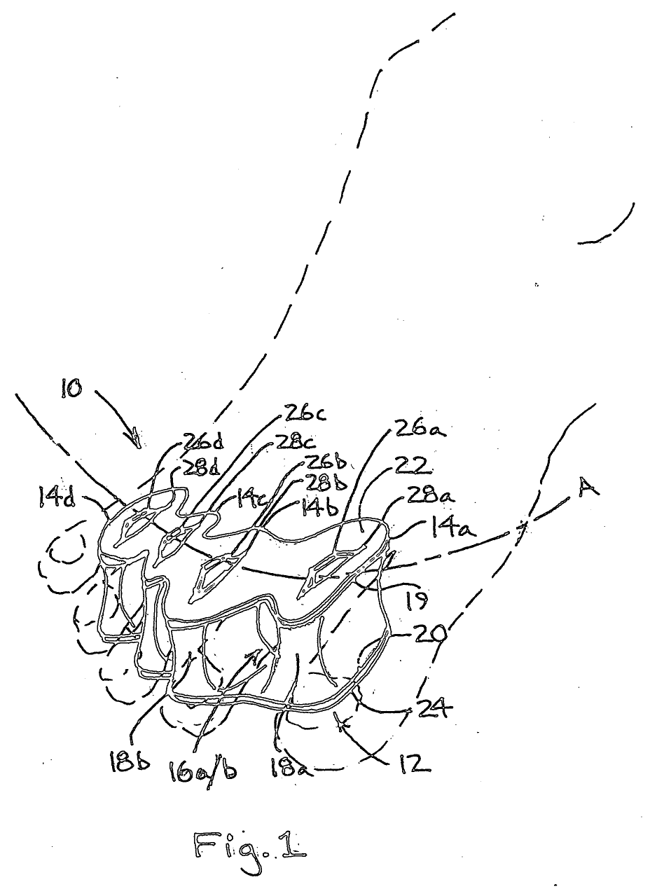

FIG. 1 shows the invented orthotic foot platform apparatus 10 in an isometric view, with the splayed toes of a human foot illustrated thereon by phantom (dashed) lines. Platform 10 may be seen to be characterized by a toe-support region 12 that is slightly depressed and also slightly downwardly, forwardly inclined, relative to the foot's arch. Toe-support region 12 in accordance with the invention includes preferably plural vertical columns 14a, 14b, 14c, 14d each configured laterally to space apart adjacent pairs of toes. Those of skill in the art will appreciate that the splayed-toe configuration of a foot fit on platform 10 corrects the problems described above that are caused by conventional, toe-confining shoe, boot and sock wear.

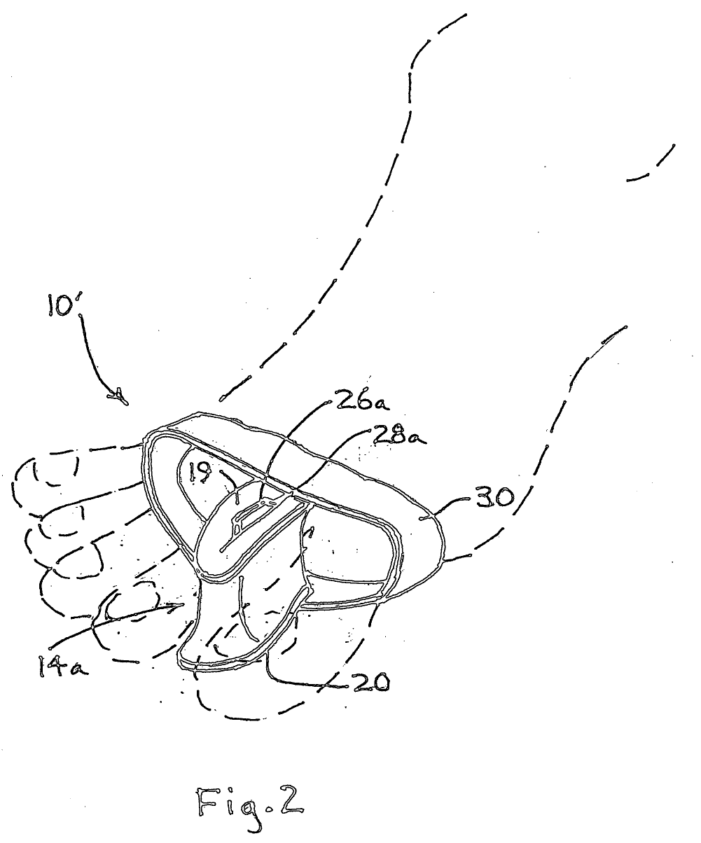

Columns 14a, 14b, 14c, 14d are similar to one another, although more significant variations are within the spirit and scope of the invention. Thus, the detailed description below of adjacent column pairs 14a/14b will be understood to describe also columns pairs 14b/14c and column pairs 14c/14d. Column pair 14a/14b may be seen to have a toe-conforming interior hollow region 16a/b for receiving a splayed toe and adjacent toe-spacing regions 18a, 18b for laterally spacing apart adjacent splayed great, second and third toes. Columns in each pair, e.g. columns 14a, 14b, are preferably made of a durable but shape-retentive material such as an elastomer, e.g. a silicone rubber or other gel polymer. Preferably, the material of the columns has a soft textured outer surface for the sake of user comfort. Of course, those of skill in the art will appreciate that any suitable material(s) may be used within the spirit and scope of the invention. For example, instead of integrally molded columns of isomorphic elastomeric material, the columns may include an interior armature of wood, plastic, ceramic, aluminum, etc. or any other suitably rigid material, preferably coated with an elastomer or other soft outer skin. Columns in each pair, e.g. columns 14a, 14b, may be seen from FIGS. 1 and 2 to arch inwardly or concavely top-to-bottom in any circumferential elevational view in a preferably smooth curve that conforms with the exterior circumferential curvature of the toe, although any suitable shape may be used that effects adjacent toe spacing.

Each column within the series of column pairs, e.g. columns 14a, 14b, may also be seen to have an upper region 19 and a lower region 20 that are generally planar and generally parallel with one another, preferably wherein upper region 19 is smaller in surface area than lower region 20, with a slight mirror-image convergence from bottom to top, as shown. Parallel planar upper and lower regions 19, 20 will be seen to mate, respectively, with a medially extending upper web structure 22 and lower web structure 24 that fix and stabilize adjacent column pairs 14a/14b, 14b/14c, 14c/14d relative to one another. Inclusion of at least one or the other of such upper and lower web structures is preferred and inclusion of both is most preferred. Preferably, the upper and lower web structures are made of the same material as are columns 14a, 14b, 14c, 14d, and, in accordance with one embodiment of the invention, the web structures are integrally molded as part of the columns 14a, 14b, 14c, 14d.

It may be seen from FIG. 1 that columns 14a, 14b, 14c, 14d in accordance with one embodiment of the invention are positioned along an arc A (a dash-dotted line) that conforms with the arc of the ball of the foot and with the distal ends, i.e. the tips, of the toes of the foot. Alternatively, and yet within the spirit and scope of the invention, columns 14a, 14b, 14c, 14d may be positioned in a straight or other curved line or other configuration that nevertheless splays the toes in accordance with the invention.

Spacer inserts or so-called ‘shims’ 26a, 26b, 26c, 26d may be seen preferably to be provided within one or more slots or slotted recesses 28a, 28b, 28c, 28d provided within central regions of columns 14a, 14b, 14c, 14d, respectively, as shown. Columns 14a, 14b, 14c, 14d with inserts 26a, 26b, 26c, 26d positioned therein may be referred to herein simply as spacers.

Inserts 26a, 26b, 26c, 26d may be of varying width predetermined for a given foot to provide the needed degree of spacing between adjacent toes. Those of skill in the art will appreciate that the degree of toe splay in accordance with the invention may be adjusted as between adjacent toes and/or over time to provide needed orthotic correction on an individual basis, rendering apparatus 10 highly adaptive to diverse users and toe configurations. Such is possible, within the spirit and scope of the invention, without requiring differing width dimensions among columns 14a, 14b, 14c, 14d. Those of skill in the art will appreciate that the elastic quality of the material from which columns 14a, 14b, 14c, 14d is made permits them to accommodate spacer inserts 26a, 26b, 26c, 26d of various widths, thereby providing proper inter-digital spacing for various individual users.

Inserts 26a, 26b, 26c, 26d may be formed of any suitable material and to any suitable dimensions, whether the same or different from one another. In accordance with one embodiment of the invention, the inserts are suitably formed of a soft and durable but shape-retentive material such as ethylene vinyl acetate (EVA) or polystyrene foam having slightly more rigidity than the material from which the columns are formed. The inserts may be made of any suitable material, e.g. EVA, polystyrene, wood, fiberboard, aluminum, etc. within the spirit and scope of the invention, and may be formed in any suitable manner, e.g. by cutting or die-stamping them from sheet material into a desired shape, as illustrated. Alternatively, but within the spirit and scope of the invention, the inserts may be molded using any suitable technique into any suitable shape and size that is organic, e.g. anatomic, to the wearer's foot. For example, the inserts may be in a V or hourglass shape that imposes more plantar or dorsal force on the toes than would one of parallel sidewall or uniform cross section. Thus the inserts effectively widen the columns into which they are inserted and relatively fix the inter-digital spacing of the toes of the user's foot.

Referring still to FIG. 1, it may be seen that columns 14a, 14b, 14c, 14d are preferably of progressively decreasing length to conform to the progressively decreasing length of the toes of the average foot (from the great toe to the fifth toe). Similarly, columns 14a, 14b, 14c, 14d are preferably of progressively decreasing width to conform to the progressively decreasing width of the toes of the average foot. Similarly, columns 14a, 14b, 14c, 14d are preferably of progressively decreasing height to conform to the progressively decreasing height of the toes of the average foot. Finally, similarly, columns 14a, 14b, 14c, 14d are preferably of progressively decreasing lateral center-to-center spacing (i.e. pitch) also to conform to the progressively decreasing width of the toes of the average foot.

Thus, on average, in accordance with one embodiment of the invention, columns 14a, 14b, 14c, 14d are of approximately 7/16 inch width, approximately one inch length and approximately 3/4 inch height, with the center-to-center spacing therebetween of approximately 1 1/8 inches. In accordance with one embodiment of the invention, spacing inserts 26a, 26b, 26c, 26d are of approximately 3/16 inch width. Those of skill in the art will appreciate that variations in these dimensions are contemplated by the invention as being within its spirit and scope to accommodate foot sizes or inter-digital spacings that are smaller or larger than average.

In accordance with one embodiment of the invention, apparatus 10 is integrally molded so that upper and lower spacer-interconnecting web structures 22, 24 are integrally and intimately and contiguously connected to columns 14a, 14b, 14c, 14d. Alternatives to such an embodiment of the invention are contemplated, however, and are within the spirit and scope thereof. For example, separately formed, e.g. molded, web structures 22, 24 may be interconnected with columns 14a, 14b, 14c, 14d by the use of an adhesive. In accordance with one embodiment of the invention, web structures 22, 24 are of approximately 3 1/2 inch width and 3/32 inch thickness. Those of skill in the art will appreciate that the thickness of web structures 22, 24 affect the facility with which apparatus 10 might be fitted within the toe box of a sock, shoe or boot while still effectively promoting toe-splaying and other orthotic advantages.

Those of skill in the art will appreciate that apparatus 10 as described and illustrated is useful in orthopedic treatment of the foot without its inclusion in the toe box of a sock, shoe or boot. The modern child's or adult's foot has, for the reasons described in the Background section, a tendency for the toes to resist spreading, i.e. to pinch together tightly into the shape of a tapered sock, shoe or boot. Thus, apparatus 10 has been discovered very effectively to be gripped in normal use between the toes when properly placed and to remain in place on the foot. The invented apparatus thus is useful in therapeutic, clinical and hospital settings or other settings in which no sock, shoe or boot wear is anticipated or desired. While remaining securely gripped by the user's toes, apparatus 10 effectively enforces a desirable splaying, or spreading, of the user's toes, obviating the requirement of any other footwear in many settings while providing comfortable orthotic correction of joint, bunion and other foot problems relatable to deformed or abnormal metatarsal joints or appendages.

Of course, it is within the spirit and scope of the invention to attach apparatus 10 to the user's foot via a suitable fastener, e.g. a loop of fabric or polymer material around the great and fifth toes. It also is within the spirit and scope of the invention to incorporate apparatus 10 within the toe box of a sock, slipper, shoe, boot or other form of footwear or orthotic foot appliance. Thus, many variations in configuration and combination are contemplated as being within the spirit and scope of the invention.

FIG. 2 illustrates another embodiment of the invented apparatus indicated as 10′. Apparatus 10′ may be seen to include only one such column 14a for splaying adjacent toes of a foot via a suitable fastener 30. Again, it is pointed out that fastener 30 is optional in many uses of apparatus 10′ due to the natural inclination of the adjacent toes of the foot to snugly grip the column. If better retention is desired, however, fastener 30 may be provided in the form of an adjustable forefoot-encircling strap that affixes apparatus 10′ to the end of the foot and ensures its retention. Preferably, the strap encircles the user's foot directly behind the proximal ends of the toes, thus avoiding inadvertent and undesirable elevation of the ball of the foot. Alternatively, fastener 30 may take the form of a woven fabric semi-stocking or sock that fits tightly over the end of the foot and helps maintain apparatus 10′ in place. Or it may take the form of an elastomer band that extends circumferentially around the end of the foot directly behind the toes. Or it may take the form of a molded elastomeric web that grips the end of the foot, preferably distal to the ball of the foot. Any such strap or web forms of fastener 30 may be suitably attached to column 14a as by a simple formed button-hole mechanism, a loop-and-pile mechanism, a simple adhesive, etc. Or it may take the form of a full-length sock that snugly fits the user's foot and yet maintains the proper position of apparatus 10′ between the desired pair of adjacent toes.

One contemplated embodiment of fastener 30 is in the form of a proximal toes-encircling strap that is integrally molded with column 14a and of the same elastomeric material, as is illustrated in FIG. 2.

Any suitable alternative configurations of fastener 30 are within the spirit and scope of the invention. It will also be appreciated that fastener 30 optionally may be used in conjunction with apparatus 10 having preferably plural, e.g. four, such columns 14a, 14b, 14c, 14d, as described and illustrated in detail above with reference to FIG. 1.

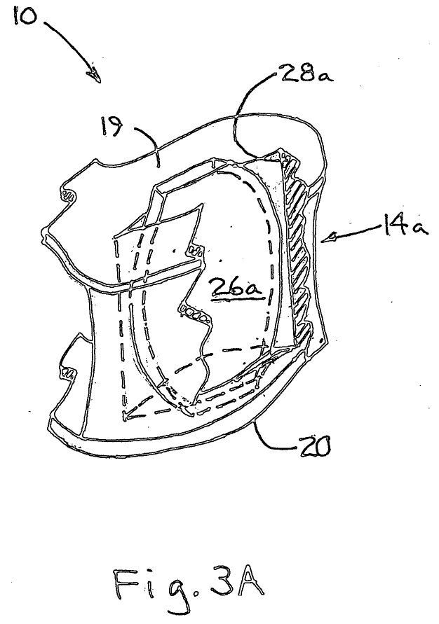

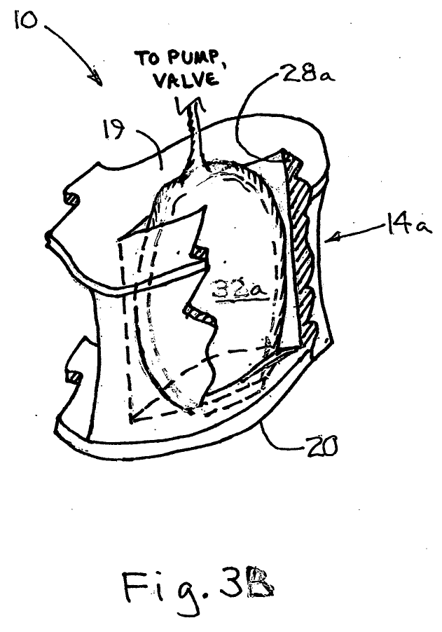

For the sake of complete detailed description of the invention, FIGS. 3A and 3B illustrate column 14a in enlarged, fragmentary, cutaway views, thereby to illustrate the detailed construction of each column described above. Referring to FIG. 3A, it may be seen that column 14a may be seen generally to be in a cylindrical shape featuring a circumferential, top-to-bottom concave shape somewhat resembling an hour glass. Centrally located slotted recess 28a may be seen in accordance with one embodiment of the invention to extend through column 14a from upper region 19 to lower region 20. Similarly, insert 26a may be seen in accordance with one embodiment of the invention to extend through column 14a from upper region 19 to lower region 20. Those skilled in the art will appreciate that, in accordance with one embodiment of the invention, recess 28a and insert 26a are shape and dimension conforming, i.e. they are isomorphic and isometric. This conformity and the choice of material therebetween ensures in accordance with one embodiment of the invention straightforward insertion and removal of the insert into and from the recess while providing excellent retention of the insert within the recess when desired for adjusting and fixing inter-digital spacing. Variations are contemplated, e.g. the slotted recess and insert therein may extend only partway through the vertical extent of the column (from upper region 18 toward lower region 20), that effectively provide spacers that preferably may be width-adjusted, e.g. by selection and insertion of an insert of desirable width, to individual user needs.

Referring briefly to FIG. 3B, it is contemplated as being within the spirit and scope of the invention to provide adjustable-width inter-digital toe spacers, or columns, as described above by any one or more alternative means. For example, an air bladder 32a inserted within slotted recess 28a of column 14a may be pumped up or down to a desired volume and pressure to adjust the width of the effective inter-digital spacing for each adjacent pair of toes. Or, within the spirit and scope of the invention, column 14a very simply may comprise nothing more than the air bladder itself extending vertically between adjacent toes, wherein the air bladder is width-adjustable to fix inter-digital spacing by increasing or decreasing the pressure of the air therein. The air bladder may have a hand- or foot-operated pump (not shown) that is used by either the orthotic device-prescribing specialist or orthotic device-wearing patient to properly adjust the inter-digital spacing. Those of skill in the allied arts will appreciate that a controllable valve (also not shown) between the pump and the bladder would permit the selective, controlled introduction or expulsion of air into and out of such a bladder. Also within the spirit and scope of the invention are hydraulic bladders functioning in a similar fashion to the pneumatic bladders described above as providing useful alternatives to the inserts described and illustrated herein as forming a part of a preferred embodiment of the invention.

Thus, the invention may be broadly characterized as involving adjustable toe spacing that meets the individualized toe reorientation of a single wearer of the orthotic device as well as the diverse toe spacing requirements of a human population that suffers foot deformation or toe alignment abnormalities.

Those of skill in the art will appreciate that the columns alone are characterized by a nominal width to enforce a nominal inter-digital spacing. One particular advantage of the invention is that the nominal width of the standard column can be increased by incorporation of an appropriate insert. Thus, a given column may be of nominal width for a given range of user needs and yet the apparatus can be customized for an individual user's needs by use of an insert of appropriate width to increase inter-digital spacing beyond the nominal spacing provided by the column alone. Moreover, the same column can accommodate various users or various toes by inclusion of an appropriate insert to produce a desired inter-digital spacing.

The provision of fewer than four spacers is contemplated by the invented apparatus. For example, one or more spacers as described above to include an insert into a hollow recess in a central region of a column may used to extend between one or more pairs of adjacent toes thus to enforce a splaying of the one or more pairs of adjacent toes. Thus, it is contemplated as being within the spirit and scope of the invention to provide as few as one spacer and as many as four spacers in apparatus 10. Those of skill in the art will appreciate that the invention thus is not in any way limited to the embodiment described and illustrated herein as including preferably four such spacers for four corresponding pairs of adjacent toes, as might be typically used for a person having five toes.

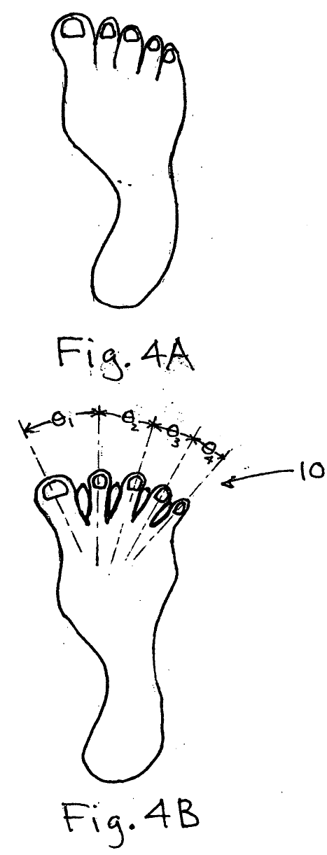

FIGS. 4A and 4B illustrate the manner in which toes are advantageously but controllably splayed by use of the invented method and apparatus. FIG. 4A shows an adult foot, without benefit of the invention, having adjacent toes typically in close and often impinging contact with one another. FIG. 4B also shows an adult foot, using apparatus 10 in accordance with the embodiment described in detail above, having adjacent toes from the great toe to the fifth toe adjustably and fixedly splayed by predetermined angles θ₁, θ₂, θ₃, θ₄. By ‘fixedly’, it will be understood that, when width-adjusted, the inter-digital spacers substantially relatively fix the toes therebetween and therearound in a desirable, splayed configuration. (Those skilled in the art will appreciate that the adjustably fixed spacing between adjacent toes is only nominal. I.e. the width of the spacing between adjacent toes may vary in response to a wearer's normal walking or running gait. Nevertheless, the nominal spacing therebetween is ‘fixed’ at a nominal spacing by virtue of the present invention.) Thus, the invented apparatus differs significantly in structure, form and function from prior art and substantially width-compressible sponge devices intended ever-so-slightly to part one's toes for the purpose of painting the toenails without incidentally smearing toenail polish on the skin of an adjacent toe.

Those of skill in the art will appreciate that these angles may be the same or different for different individual users and for different toe configurations for a given individual user. This is one of the excellent advantages of the invention, which facilitates widening by a desired amount the width of the inter-digital spacers by installing a properly dimensioned shim within the recesses provided therein. Thus, apparatus 10 (or alternatively apparatus 10′) preferably made from a single, common mold nevertheless is made to accommodate individual users and individual toes thereof by the simple expedient of adjusting the inter-digital spacing before, during or after apparatus 10 (or apparatus 10′) of nominal spacing is fitted to the user's foot. Those skilled in the art will appreciate that alternative and still within the spirit and scope of the invention portions of apparatus 10 may be made from two or more molds and adhered or otherwise joined.

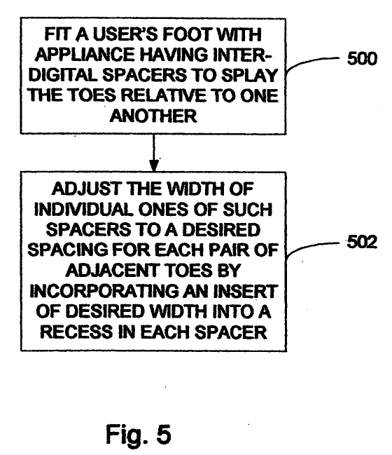

FIG. 5 shows is a flowchart illustrating an orthotic foot care method in accordance with another embodiment of the invention. The foot care method includes a) fitting a user's foot with an appliance having spacers for extending between pairs of adjacent toes and spreading the same relative to one another at 500 and b) adjusting the width of individual ones of such spacers to a desired spacing for each pair of adjacent of toes on the user's foot at 502. Preferably, the appliance is as described above with respect to apparatus 10 and the width adjustment is as described above with respect to incorporation in each column of a variable width insert to adjust the overall width of what are referred to herein as spacers. Those of skill in the art will appreciate that the fitting and adjusting may be performed in either order, i.e. the appliance may be adjusted and then fit, or it may be fit and then adjusted, or it may be coarsely adjusted and then fit and then finely adjusted, etc. All suitable variations are contemplated as being within the spirit and scope of the invented orthotic foot care method.

Those of skill in the art will appreciate that fitting may include therapeutic evaluation and metrics, e.g. X-rays or range-of-motion studies, as well as other medical diagnostic and prescriptive techniques that are known to those of skill in the art of podiatric medicine and/or therapy. As described and illustrated above, fitting appliance 10 or 10′ to the user's foot involves little more than measuring and fitting the user's affected foot and configuring appliance 10 or 10′ of the proper nominal size and number of spacers, i.e. one or more of columns 14a, 14b, 14c, 14d incorporating one or more corresponding inserts 26a, 26b, 26c, 26d. Also as described and illustrated above, adjusting the width of individual ones of such spacers involves little more than determining whether such inserts need to be narrower or wider and adjusting their width as needed to fit the user's individual prescription. Such adjusting may involve trial and error for best fit and comfort over a regimen of physical therapy or normal walking, tread-milling or running, for example. Other foot care may be performed at the same time. For example, socks, shoes or boots may be fitted with apparatus 10 or 10′ as part of the therapy. Thus, alternative or supplemental foot care steps are contemplated as being within the spirit and scope of the invention.

Finally, those of skill in the art will appreciate that the invented method and apparatus described and illustrated herein may be implemented in any suitable alternative configuration, structure or material. In accordance with one embodiment of the invention, a foot appliance and therapeutic method are described as involving the use of one or more spacers including an inter-digital column and a rigidizing and widening insert extending therein respectively made of a soft, durable, shape-retentive polymeric materials of sufficient hardness to urge and hold apart in predetermined width (or angular) relationship one or more pairs of adjacent toes. Alternative embodiments are contemplated in which each of the inter-digital columns is width-adjustable, however, including, for example, the pressurizable-bladder alternative described above. Any and all suitable alternatives are within the spirit and scope of the invention.

Having described and illustrated the principles of the invention in a preferred embodiment thereof, it should be apparent that the invention can be modified in arrangement and detail without departing from such principles. I claim all modifications and variation coming within the spirit and scope of the following claims.

1. An orthotic toe spacer comprising:
an inter-digital column for extending generally vertically between adjacent toes of a user,
the column being width-adjustable to form an orthotic spacer for urging laterally apart the adjacent toes that the column extends between to a defined inter-digital spacing.

2. The spacer of claim 1, wherein the column includes a width-adjustable recess therein for receiving an insert of defined width to fix the inter-digital spacing.

3. The spacer of claim 1, wherein the column includes a width-adjustable, pressurizable bladder for receiving a fluid under pressure to fix the inter-digital spacing.

4. An orthotic toe spacer comprising:
an inter-digital column for extending generally vertically between adjacent toes of a user, the column having a generally cylindrical hour-glass shape including cross-sectionally concave sidewalls;
a hollow elongate generally planar recess formed in a generally central region of the column; and
an insert fitted into the recess and fixedly gripped thereby, the insert rigidizing the column and laterally widening the recess and the column into which the insert is fitted;
the column having the insert within the recess forming an orthotic spacer for urging laterally apart the adjacent toes that the spacer extends between to a predetermined inter-digital spacing.

5. The spacer of claim 4, wherein the recess is dimensioned to accommodate therein an insert in the form of a shim selected from a group of shims of various widths.

6. The spacer of claim 4, wherein the insert is a pressure-adjustable pneumatic bladder.

7. The spacer of claim 4, wherein the insert is a pressure-adjustable hydraulic bladder.

8. The spacer of claim 4, wherein the column is formed of an elastomer.

9. The spacer of claim 8, wherein the insert is formed of a polymer.

10. The spacer of claim 4, wherein the column is elongated for extending along an axis between the adjacent toes and wherein the column is dimensioned in length approximately equal to the average length of the adjacent toes.

11. The spacer of claim 4, wherein the insert is of a generally circular disk shape having opposing tabs extending radially from a center.

12. The spacer of claim 4, wherein the recess extends through the column from a top region to a bottom region of the column.

13. The spacer of claim 4, wherein the recess is of a generally circular disk shape and wherein the insert is of a generally circular disk shape, wherein the recess and the insert are generally isometric and isomorphic to one another.

14. The spacer of claim 4 which further comprises:
a fastener for attaching to the foot of the user the column with the insert therein.

15. Orthotic foot platform apparatus comprising:
two or more inter-digital spacers for extending between one or more pairs of adjacent toes, each of the two or more spacers being made of a material that is shape-retentive and sufficiently durable to urge apart and maintain a predetermined separation between adjacent toes of the one or more pairs, each of the spacers having upper and lower generally parallel planar regions, and
one or more interconnecting web structures fixedly attached to and extending across the upper and lower regions of the two or more spacers to fix the spacers relative to one another in a generally parallel configuration to produce a toe channel between the two or more spacers for receiving one of the two or more adjacent toes and for spacing apart at least one other of the one or more pairs of adjacent toes.

16. The apparatus of claim 15, wherein each of the two or more spacers is width-adjustable to fix inter-digital spacing.

17. The apparatus of claim 15, wherein each of the spacers includes a recess therein extending inwardly from at least one of the upper and lower regions, which apparatus further comprises:
one or more inserts within the one or more recesses, the inserts configured to rigidize the spacers and the inserts dimensioned to widen the recess and the column thereby to fix inter-digital spacing between the adjacent toes of the one or more pairs.

18. The apparatus of claim 17, wherein the apparatus comprises four of the spacers having four recesses therein and four of the inserts within the four recesses.

19. The apparatus of claim 18, wherein the four spacers and the one or more web structures are formed together by integrally molding the same.

20. The apparatus of claim 18, wherein the four spacers and the one or more web structures are formed of an elastomeric material.

21. The apparatus of claim 20, wherein the inserts are formed of an elastomeric material having a defined width that fixes a desired inter-digital spacing.

22. The apparatus of claim 20, wherein the inserts include an adjustably pressurizable bladder having a defined width that fixes a desired inter-digital spacing.

23. The apparatus of claim 20, wherein the four spacers are adhered at the respective upper and lower regions thereof to the one or more web structures.

24. The apparatus of claim 23, wherein the one or more web structures are curved in an arc conforming to an arc of the toes and fix the four spacers in an arc corresponding thereto.

25. The apparatus of claim 24, wherein the four recesses extend through the four columns from the top region toward the bottom region thereof, and wherein the four recesses are dimensioned to accommodate therein shims selected from a group of shims of various widths.

26. The apparatus of claim 23 which further comprises:
a fastener for attaching to the foot of the user the integrally molded web structures and spacers having the four inserts therein.

27. An orthotic foot care method of, the method comprising:
fitting a foot of a user with an appliance having spacers for extending between pairs of adjacent toes and for spreading the same relative to one another, and
adjusting the width of individual ones of such spacers to a desired spacing for each pair of adjacent toes on the foot of the user.

28. The method of claim 27, wherein the adjusting includes selecting and inserting shims of predetermined width into corresponding recesses formed within the bodies of the spacers.

29. The method of claim 27, wherein the adjusting includes selecting and inserting pressurizable bladders of adjustable width into corresponding recesses formed within the bodies of the spacers.

30. The method of claim 27 which further comprises:
fastening the appliance to the foot of the user to secure it.
